(19) United States
(12) Patent Application Publication
(10) Pub. No.: US 2008/0287992 A1
(43) Pub. Date: Nov. 20, 2008

(54) Surgical method for suturing soft tissue to a bone, in particular the rotator cuff to the humerus

(75) Inventors: Alain TORNIER, Saint Ismier (FR); Gilles WALCH, Lyon (FR); Pascal BOILEAU, Nice (FR)

Correspondence Address: Ralph A. Dowell of DOWELL & DOWELL P.C., 2111 Eisenhower Ave, Suite 406, Alexandria, VA, 22314, US

(73) Assignee: PHUSIS, Saint Ismier (FR)

(21) Appl. No.: 12/149,756

(22) Filed: May 7, 2008

(30) Foreign Application Priority Data
May 9, 2007 (FR) 07 03311

(51) Int. Cl. A61B 17/04; A61B 19/00

(52) U.S. Cl. 606/232; 128/898

(57) ABSTRACT

The aim of this method is to improve the efficiency and ease of implanting a means for suturing a soft tissue to a bone, while allowing it to be carried out by arthroscopy in particular. In accordance with the method according to the invention:
a flexible closed link is provided, which is attached in a non-detachable manner at a first segment of the periphery thereof belonging to a first part of the link to an attachment means supported by an anchor,
the anchor is engaged in a bone region adjacent to a bone region underneath the soft tissue to be sutured,
a second part of the link, which is distinct from the first part of the link, is passed a plurality of times through the soft tissue in such a way that at least one portion of the second part of the link runs on a face of the soft tissue turned the opposite way to the anchor,
the link is fastened, at a second segment of the perimeter thereof belonging to the second part of the link, to a fastening means supported by the anchor, the link thus drawing the soft tissue tight, and
the link is again fastened, at least one third segment of the perimeter of said link belonging to the at least one portion of the second part of the link running on the soft tissue, to the fastening means, the link thus drawing the soft tissue even tighter.

The present invention relates to a surgical method for suturing soft tissue to a bone.

This type of suture system is implanted to restore the continuity between soft tissue, such as a muscle, a tendon or a ligament and a bone structure, the anatomical connection initially connecting said tissue and said bone structure having been damaged, or even severed, due to a disease or a trauma. The invention is thus particularly applicable for repairing and fixing the rotator cuff, i.e. the tendinous structure formed by the four rotator muscles of the shoulder at the upper end of the humerus in a human patient.

The cuff is generally sutured using filament threads which are passed through the cuff before being fastened to the humerus, either directly by producing transosseous points, or indirectly by passing the suture threads into the proximal eyelet of an anchor, the distal portion of which having previously been fixed in the humeral bone. The quality of the suture thus produced is often found to be poor: in the case of the transosseous suture points, the suture threads frequently penetrate the humeral bone matter, by cutting into said bone matter, causing the fastening of the cuff to the humerus to become loose. This drawback is all the more noticeable when the bone matter is of a poor quality, which is frequently the case in elderly patients suffering from osteoporosis. In addition, when a suture anchor is used, its anchoring in the bone may be weak when the spongy bone in which the anchor is fixed is of a poor quality. In this case, the surgeon generally inserts the anchor more deeply into the humerus which leads to the proximal eyelet of the anchor being drawn into the interior of the humeral bone envelope with a significant risk of the suture threads subsequently cutting into the surrounding bone matter as a consequence.

In addition, the sutures made at a joint, such as the shoulder, are generally carried out by arthroscopy, using, in particular, one or more arthroscopy cannulae, with which the surgeon examines the joint region to be treated and operates in said region. In practice, the surgical movements are complex, since they substantially consist in controlling the instruments, which are introduced and slid into said cannula(e) and are, for example, 140 mm in length and have an inner diameter of only 8 mm. Knotting the suture threads under these conditions is a delicate and tedious operation and does not guarantee that the knots formed are sufficiently tight.

The object of the present invention is to propose a suturing method which, while being able to be carried out by arthroscopy, is simpler and more effective and avoids having suture threads to be knotted.

For this purpose, the invention relates to a surgical method for suturing soft tissue, such as a muscle, a ligament or a tendon, to a bone, in which the following steps are carried out successively:

providing a flexible closed link which is attached in a non-detachable manner at a first segment of the periphery thereof belonging to a first part of the link to an attachment means supported by an anchor,

engaging the anchor in a bone region adjacent to a bone region underneath the soft tissue to be sutured,

passing a second part of the link, which is distinct from the first part of the link, a plurality of times through the soft tissue in such a way that at least one portion of the second part of the link runs on a face of the soft tissue turned the opposite way to the anchor,

fastening the link, at a second segment of the perimeter thereof belonging to the second part of the link, to a fastening means supported by the anchor, the link thus drawing the soft tissue tight, and

again fastening the link, at least a third segment of the perimeter of said link belonging to the at least one portion of the second part of the link running on the soft tissue, to the fastening means, the link thus drawing the soft tissue even tighter.

The idea on which the invention is based is to dispense with producing knots in the suture thread by simultaneously connecting an attachment means and a fastening means of a flexible closed link to a suture anchor in such a way that, in use, the link is connected to the bone in a fixed manner at least three distinct segments around its perimeter via the anchor, at least two of said at least three segments belonging to a part of the link which was previously passed through the soft tissue to be sutured. When the anchor is fastened to the second segment of the link whilst the first segment is attached to said anchor, the link, which has been passed through the soft tissue, draws said soft tissue towards the bone region in which the anchor is attached by drawing this tissue so as to rest against the bone. This tensioning is increased when the or each third segment is fastened to the anchor at the same time as the second segment is fastened. The tensioning is prevented from becoming loose due to the attachment and fastening means of the link acting on the first and the second and third segment(s) respectively. Therefore, in the particular case of suturing the rotator cuff, the surgeon connects, in a first step, the anchor to the upper end of the humerus, the lateral external side of said end in particular, then, in a second step, fastens a part of the link, which was previously passed through the rotator cuff, to the anchor, thus drawing the cuff tightly against the upper face of the end of the humerus. Due to the fact that the link is fixed at least three distinct segments thereof to the humerus, this link keeps the cuff tight. The surgical movements required to implant the suture system according to the invention are thus rapid and simple. They may also be carried out by arthroscopy without impairing the quality of the suture produced, it of course being understood that the surgical operation of implanting the system may also be carried out in a primarily “conventional” method with a larger incision and drawing the shoulder tissue towards the exterior.

By adjusting the active length of the link in a predetermined manner relative to the soft tissue to be sutured, as a function of the thickness of said tissue, the invention may be used to suture tissue of variable thickness without requiring any particular precautionary measures to be taken during manipulation. In addition, the anchoring depth of the anchor in the bone is in practice chosen freely by the surgeon without causing any problems in subsequent operations to manipulate the system according to the invention, since the link, in particular at the second and third segments, extends sufficiently in terms of length to the anchor to which it is attached in the region of its first segment. The mechanical performance of the suture obtained by the method according to the invention is thus improved.

A clearer understanding of the invention will be facilitated by reading the following description given solely by way of example and described in reference to the drawings, in which:

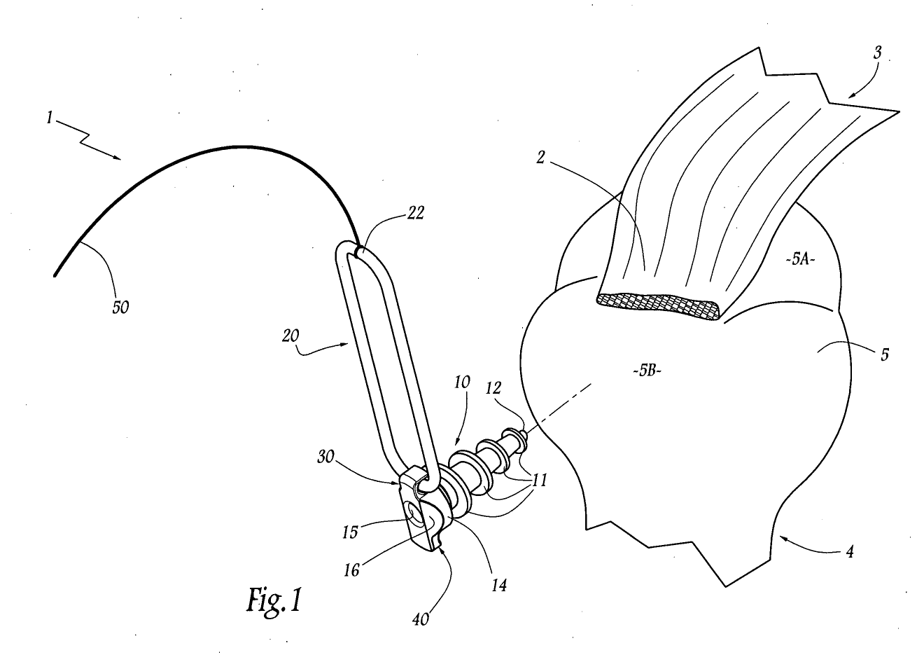

FIG. 1 is a schematic perspective view of a system to be implanted according to the invention which has not yet been implanted and is associated with the upper part of a human humerus;

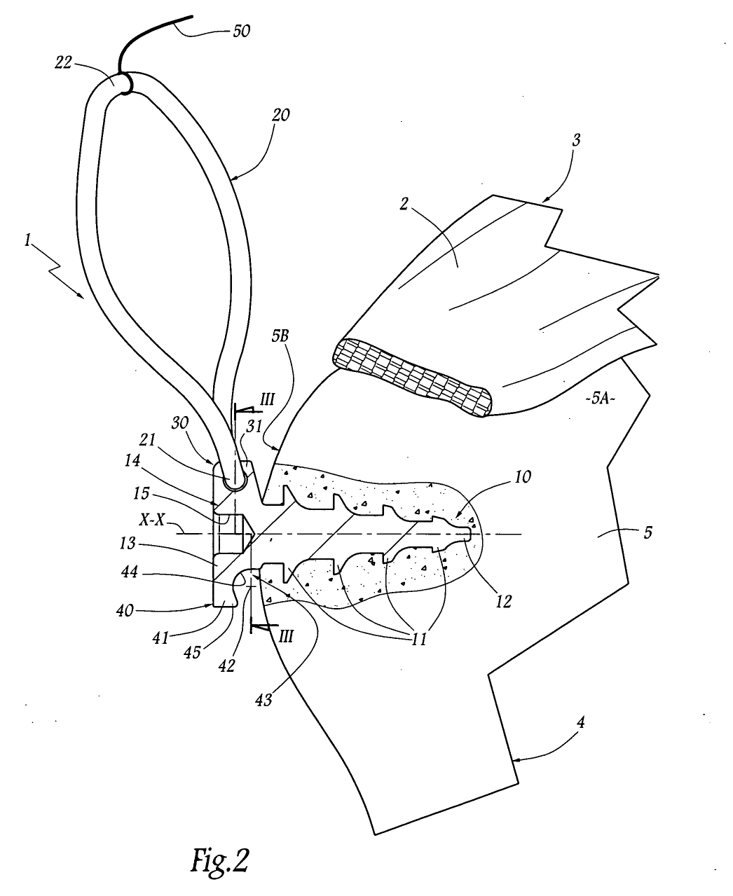

FIG. 2 is a partial longitudinal sectional view of the system of FIG. 1 while being implanted in the region of the upper part of the humerus, viewed in elevation in a generally anteroposterior direction, partially cut away;

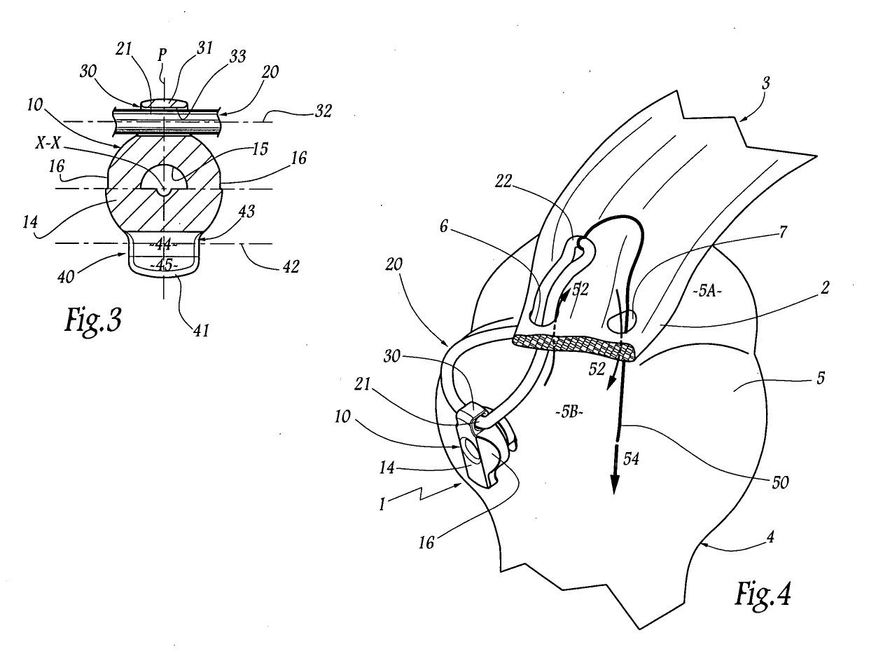

FIG. 3 is a section along the line III-III of FIG. 2;

FIGS. 4, 5A and 6A are schematic views similar to FIG. 1, respectively illustrating the successive steps of implanting the system of FIG. 1, subsequent to the step shown in FIG. 2; and

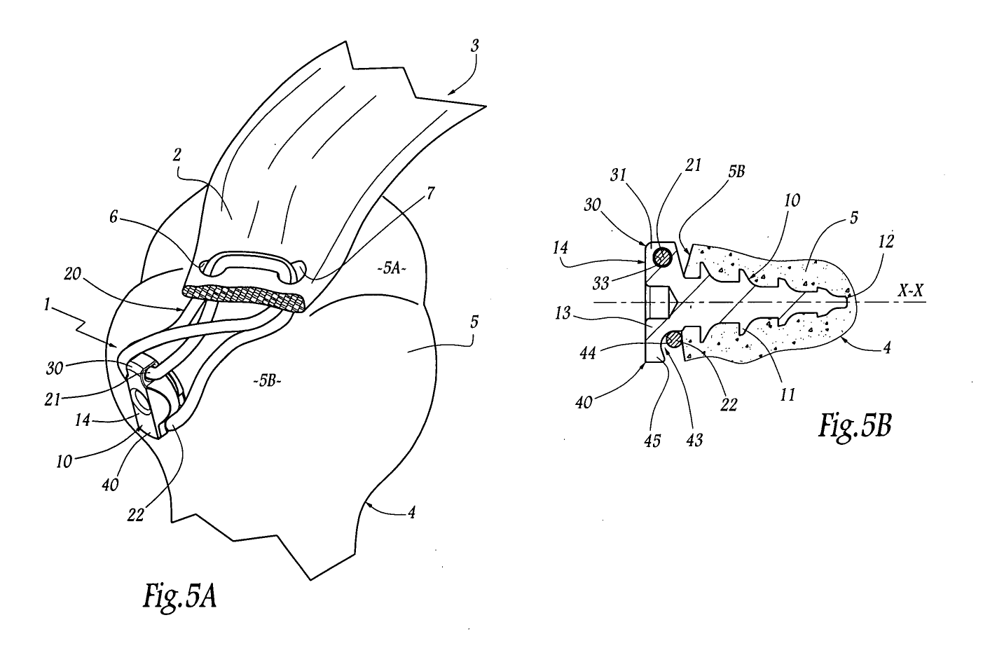

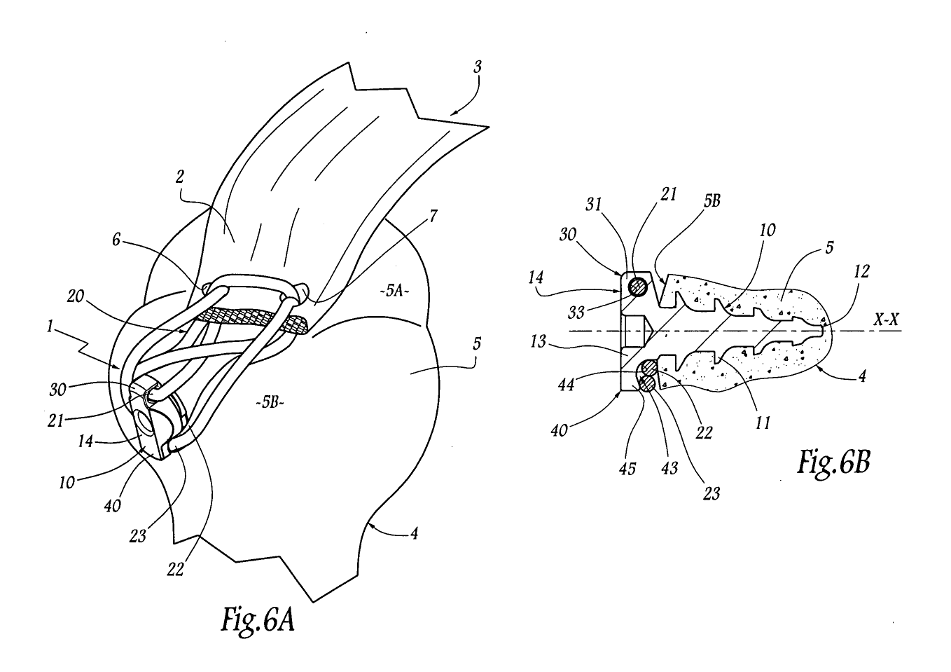

FIGS. 5B and 6B are sections in the same plane as that of FIG. 2, showing the systems in the configurations of FIGS. 5A and 6A respectively.

In the figures, a system 1 for suturing the cuff 2 of the rotator muscles 3 to the humerus 4 of a human being is shown, only the upper part of which humerus can be seen in the figures. The cuff 2 is the tendinous structure at the end of the four rotator muscles 3 in a shoulder. In the non-damaged anatomical configuration of this tendinous structure, it is attached and connected securely on the upper end 5 of the humerus 4, more specifically on the upper face 5A of the end humeral tuberosities, external and anterior, namely on the upper face of the greater tuberosity of the humerus and the lesser tuberosity of the humerus. The aim of the system 1 is to restore continuity between the rotator cuff and the humeral end 5 in order to treat damage, even when the normal anatomical connection has been severed.

The terms “proximal” and “distal” are consequently defined relative to the direction in which the suture system 1 is implanted in the humerus 4, the term “distal” thus indicating a direction generally towards the right in all of the figures with the exception of FIG. 3.

The suture system 1 comprises an anchor 10 which extends longitudinally about a proximodistal central axis X-X. On its outer face, this anchor is provided with peripheral ribs 11 which are centred on the axis X-X and have diameters which increase in a regular manner from the distal end 12 of the anchor towards the proximal end 13 thereof. Said ribs are adapted to be engaged directly with the spongy bone of the upper end 5 of the humerus 4 by being impacted in said spongy bone by means of insertion by force along the axis X-X.

For this purpose, the proximal end 13 of the anchor 10 is shaped as a generally cylindrical head 14 which has a circular base and is centred on the axis X-X. Said head 14 has a recess allowing the anchor to be impacted using a particular instrument (not shown) provided with a recess corresponding to the aforementioned recess. In the example shown in the figures, said aforementioned recess comprises a hole 15 which is coaxial with the axis X-X and opens onto the proximal face of the head 14.

For reasons provided below, the proximal face of the head 14 is advantageously provided with two inclined surfaces 16 which diverge from one another in the direction of the distal end 12 of the anchor, both diverging in a symmetrical manner relative to a plane P which contains the axis X-X and corresponds to the sectional plane of FIG. 2.

The system 1 also comprises a closed flexible link 20 forming, completely by itself, a loop closed on itself. In the example shown in the figures, said link has a substantially circular cross-section in such a way that said link is similar to a loop formed by a monofilament.

In practice, the link 20 is intended to draw the cuff 2 of the rotators 3 tight, as described below, in such a way that, around its perimeter, said link is not resilient or, at the very least, is markedly less resilient than the cuff 2.

When in use, the link 20 is attached in a permanent manner to a proximal end 13 of the anchor 10 by an attachment means 30 supported by the head 14. This attachment means 30 comprises a tubular wall 31 which is centred on a longitudinal geometrical axis 32 extending in a direction which is orthoradial to the axis X-X, as can be seen in FIG. 3. The wall 31 thus internally delimits a hole 33 passing completely through the attachment means 30 along the axis 32. This hole 33 has a cross-section which substantially complements that of the link 20 in such a way that said hole is sized so as to adjustably receive the link which is thus attached in a non-detachableable manner to the anchor 10 at a segment 21 of the perimeter thereof.

In practice, a number of technical options are possible to connect the link 20 and the attachment means 30 in a non-detachable manner. Therefore, before the suture system 1 is put into operation, the link 20 may, for example, be open and threaded, via one of its free ends, into the hole 33, then closed on itself by knotting the two ends. Another possibility is to provide that a part of the wall 31 is detachable relative to the rest of said wall so as to be able to reach, particularly in a radial manner, the hole 33 to place the segment of the link 21 at that location.

In addition, in the example shown in the figures, the wall 31 is integral with the anchor 10 in the region of the head 14 of said anchor. In a variant not shown, the wall 31 and the head 14 may be composed of parts formed independently of one another then assembled securely with one another by any biocompatible means which guarantees that the parts are connected to one another permanently during use.

Diametrically opposite the attachment means 30 relative to the axis X-X, the anchor 10 is provided with a means 40 for fastening the link 20. This fastening means 40 is supported by the head 14 and comprises a substantially semi-tubular wall 41 which extends lengthwise about a geometrical axis 42 which is orthoradial to the axis X-X and is open on the distal side of the anchor while being closed on the proximal side of the anchor. The wall 31 of the attachment means 30 and the wall 41 of the fastening means 40 each extend lengthwise in the region of respective opposing portions of the outer periphery of the head 14 so as to project towards the exterior from the lateral face of the generally cylindrical body of said head.

In the embodiment shown in the figures, the inclined surfaces 16 of the head 14 extend, along the periphery of the head, out of the walls 31 and 41 in such a way that these walls 31 and 41 thus each extend either side of the plane P, this plane advantageously forming a plane of symmetry for each of the two walls, as is clearly shown in FIG. 3.

The semi-tubular wall 41 internally delimits an aperture 43 which extends along the axis 42 and which opens onto the distal side of the anchor 10. When in use, as explained in detail hereinafter, the aperture 43 is intended to receive a longitudinal segment of the link 20 in such a way that the surface 44 of the wall 41 delimiting said aperture is advantageously substantially semi-cylindrical about the axis 42 with a radius of curvature greater than or equal to that of the cross-section of the link 20.

At its part furthest away radially from the axis X-X, the wall is shaped as an edge 45 which borders in a projecting manner the peripheral region corresponding to the aperture 43. In the embodiment shown, the distal surface of the edge 45 generally extends into the extension of the semi-cylindrical surface 44 and becomes more distant from the axis X-X in such a way that, along the axis X-X, a portion of said surface 44 is located proximally set back from the distal end of the edge 45, as can be clearly seen in the section shown in FIG. 2. It is thus to be understood that, when a segment of the link 20 is received in the aperture 43, the edge 45 holds the link segment in the sense that said link segment can only be released from the aperture 43 by being sufficiently distant along the axis X-X from the surface 44 in the direction of the distal end 12 of the anchor 10 so as to be able to pass around the distal end of the edge 45.

Implantation of the suture system 1 will now be described in the context of an arthroscopic surgical operation.

In a first operation step, a surgeon makes one or more mini incisions in the region of the patient's shoulder and subsequently introduces arthroscopic instruments therein, in particular one or more arthroscopic cannulae in the form of relatively long flexible tubes, generally 140 mm in length, each having a small interior diameter of generally 8 mm. With a cannula of this type, the surgeon can examine the bone region for implanting the suture system 1, namely the upper end 5 of the humerus 4, as well as the state of the rotator cuff 3 to be sutured to said humeral end 5, with an arthroscopic camera in particular which is inserted and slid in the or one of the aforementioned cannula(e) until the distal end of said cannula.

In a second operation step, the surgeon conveys the system 1 to the vicinity of the cuff 2 by inserting it and sliding it in the or one of the aforementioned arthroscopy cannula(e) to the distal end of said cannula. The surgeon then impacts the anchor 10 in the spongy bone forming the humeral end 5 by engaging the ribs 11 in the lateral external face 5B of said humeral end, as shown in FIG. 2. For this purpose, the surgeon uses an arthroscopic impaction instrument (not shown), the distal end head of which cooperates with the proximal head 14 of the anchor, in particular by complementing shapes, in order to insert said anchor with force into the humerus along the axis X-X. The surgeon adjusts the depth to which the anchor is anchored in the bone at his convenience as a function of the quality of the bone matter forming the humeral end 5 in particular.

The suture system 1 is thus configured as shown in FIGS. 2 and 3. In particular, the closed link 20 is attached to the anchor 10 by cooperation between the segment 21 thereof and the attachment means 30.

In a third operation step, the surgeon works on the link 20, in particular on a segment 22 of said link which is the most remote from the segment 21 along the perimeter of the link. For this purpose, the suture system 1 optionally comprises a thread 50 which is connected to said link segment 22 by being knotted around said link, for example. In practice, it is easier to pick up and pull the thread 50 by an appropriate arthroscopic instrument controlled by the surgeon. Therefore, as shown in FIG. 4, after having placed the cuff 2 satisfactorily on the upper face 5A of the humeral end 5, the surgeon threads, if necessary, the free end of the thread 50 successively into two adjacent holes 6 and 7 which pass completely through the cuff 2 from its lower face to its upper face, said holes advantageously being formed by a perforating needle (not shown) provided at the free end of the thread 50. As indicated by the arrows 52 in FIG. 4, the end of the thread 50 is introduced into the hole 6 from the lower face of the cuff so as to exit therefrom at the upper face, and is then introduced into the hole 7 from the upper face of the cuff so as to exit therefrom at the lower face. By subsequently pulling the thread 50, as shown by the arrow 54 in FIG. 4, the surgeon threads a part of the link 20, which is remote from the attachment means 30 and includes the segment 22 in particular, through the holes 6 and 7 successively following the same path as the thread 50.

In a fourth operation step, the surgeon fastens the link 20 to the anchor 10 by cooperation between the link segment 22 and the fastening means 40. For this purpose, he pulls the part of the link 20 emerging from the hole 7 on the lower side of the cuff so as to engage the loop which forms said link around the proximal head 14 of the anchor in such away that the segment 22 is received in the aperture 43 after having been passed over the edge 45. The surfaces 16 facilitate the link segment 22 sliding into the aperture 43, the incline of these surfaces effectively guiding and spreading out the link segment around the head 14 on either side of the wall 41. The link segment 22 thus rests against the surface 44 of the aperture 43 and is held in this aperture by the edge 45. The thread 50 is subsequently withdrawn by being cut or unknotted. The suture system 1 is thus configured as shown in FIGS. 5A and 5B. In this configuration, the link 20 is immobilised relative to the humerus 4 at the segments 21 and 22 thereof by the attachment means 30 and the fastening means 40 respectively carried by the anchor 10. In this way, the link 20 exerts traction on the cuff 2 of the rotators 3 in the region of the holes 6 and 7 so as to draw the cuff tight and to hold it so as to press against the upper face 5A of the humeral end 5. The intensity of the tensioning of the cuff relative to the humerus is assessed by the surgeon visually by an arthroscopy camera and/or by the resistance felt when fastening the link segment 22 around the means 40.

In a fifth operation step, which is carried out when the surgeon judges that the tensioning of the cuff 2 is insufficient, the surgeon picks up a part of the link which runs against the upper face of the cuff between the holes 6 and 7, and pulls said part of the link in the direction of the head 14 of the anchor 10 until it is fastened about the means 40 at a segment 23 of said part of the link, as shown in FIGS. 6A and 6B. The surgeon thus increases the tension of the suture between the cuff 2 and the humeral end 5 finely and precisely.

The surgical movements allowing the link segment 23 to be fastened relative to the anchor 10, in particular around the proximal head 14 thereof, are substantially analogous to those used for fastening the link segment 22. Therefore, in the configuration shown in FIGS. 6A and 6B, the aperture 43 receives the segments 22 and 23 simultaneously, which when considered together, are jointly held against the surface 44 while being retained in the aperture by the edge 45.

In order to allow the surgeon to further precisely adjust the tension of the suture of the cuff 2, a variant of the method involves, during the third operation step, passing the part of the link 20 remote from the attachment means 30 through the cuff more than twice, for example six times. In this case, three distinct pairs of portions of the link 20 run against the upper face of the cuff in such a way that, during the fifth operation step, the surgeon has the option of fastening one, two or three segments, in addition to the segment 22, around the means 40, thus correspondingly increasing the extent to which the cuff is tensioned by the system 1. Of course, the aperture 43 is consequently sized for this purpose in order to simultaneously receive different segments.

Various amendments and variants of the suture system 1 and of the surgical method allowing said system to be implanted are also possible. By way of example:

the attachment means 40 may extend along the periphery of the head 14 so as to more closely follow the lateral face of said head; in particular, the geometrical axis 42 of the wall 41 may extend in a direction peripheral to the axis X-X;

in the example shown in the figures, the anchor 10, the attachment means 30 and the fastening means 40 are formed in a single piece composed of a plastics material or also of other rigid materials, in particular metal materials; in a variant, this anchor, attachment means and/or fastening means may all or in part be composed of initially separate parts and are then assembled together; in addition, the material(s) forming these components may be provided so as to be resorptive;

the anchor 10 may be connected to the humerus 4 by screwing in said anchor rather than impacting it; in this case, the recess of the head 14 is provided so as to cooperate with an instrument for rotationally driving the anchor on itself about its axis X-X;

a plurality of suture systems 1 may be implanted adjacent to one another on the lateral external face 5B of the humeral end 5 so as to provide other links 20 for tensioning the cuff in a plurality of regions distributed in an anteroposterior direction; and/or

the suture system 1 may be implanted in regions other than the shoulder so as to suture soft tissue other than the cuff 2 of the rotators 3, said soft tissues being able to be ligaments or muscular structures as well as tendons; in this case, when the implantation site is not a joint, the suture system according to the invention has the advantage of being able to be implanted by endoscopic surgery.

1. Surgical method for suturing soft tissue, such as a muscle, a ligament or a tendon, to a bone, in which the following steps are carried out successively:
providing a flexible closed link which is attached in a non-detachable manner at a first segment of the periphery thereof belonging to a first part of the link to an attachment means supported by an anchor,
engaging the anchor in a bone region adjacent to a bone region underneath the soft tissue to be sutured,
passing a second part of the link, which is distinct from the first part of the link, a plurality of times through the soft tissue in such a way that at least one portion of the second part of the link runs on a face of the soft tissue turned the opposite way to the anchor,
fastening the link, at a second segment of the perimeter thereof belonging to the second part of the link, to a fastening means supported by the anchor, the link thus drawing the soft tissue tight, and
again fastening the link, at least one third segment of the perimeter of said link belonging to the at least one portion of the second part of the link running on the soft tissue, to the fastening means, the link thus drawing the soft tissue even tighter.

2. Method according to claim 1, wherein the anchor is engaged by impacting it into the bone region adjacent to the bone region underneath the soft tissue to be sutured.

3. Method according to claim 1, wherein the anchor is engaged by screwing it into the bone region adjacent to the bone region underneath the soft tissue to be sutured.

4. Method according to claim 1, wherein, when the anchor is engaged in the bone region adjacent to the bone region underneath the soft tissue to be sutured, the depth to which the anchor is anchored in the bone is adjusted freely, independently of the other steps of the method.

5. Method according to claim 1, wherein, in order to pass the second part of the link through the soft tissue, a thread is connected to the second segment, the free end of said thread is threaded successively into at least two adjacent holes which pass completely through the soft tissue, the thread is then pulled in order to thread the second part of the link through the adjacent holes successively.

6. Method according to claim 5, wherein a perforating needle is provided on the free end of the thread and wherein the adjacent holes are formed with said needle.

7. Method according to claim 1, wherein the attachment means and the fastening means are supported by a proximal end of the anchor and are located respectively in the region of the opposing portions of the outer periphery of said proximal end, and wherein the link is fastened to the fastening means by engaging the second segment then the at least one third segment around the proximal end of the anchor until said second segment then the at least one third segment is received in an aperture which is delimited by the fastening means and opens onto the distal side of the anchor.

8. Method according to claim 7, wherein, in order to receive the second segment then the at least one third segment in the aperture, said second segment then the at least one third segment are slid against inclined surfaces which are delimited by the proximal end of the anchor and which diverge from one another in the direction of the distal end of the anchor in such a way that the inclined surfaces guide and spread out the second segment then the at least one third segment on both sides of the fastening means along the outer periphery of the proximal end of the anchor.

9. Method according to claim 1, wherein the rotator cuff is thus sutured to the upper end of a humerus of a human being by engaging the anchor in the lateral external side of the end of the humerus and drawing, by means of the link, the rotator cuff tight against the upper face of the end of the humerus.

10. Method according to claim 1, implemented by means of endoscopy.

11. Method according to claim 1, implemented by means of arthroscopy.
